(19) United States
(12) Patent Application Publication
(10) Pub. No.: US 2014/0296690 A1
(43) Pub. Date: Oct. 2, 2014

(54) OBJECT INFORMATION ACQUIRING APPARATUS AND OBJECT INFORMATION ACQUIRING METHOD

(71) Applicant: CANON KABUSHIKI KAISHA, Tokyo (JP)

(72) Inventors: Takuro Miyasato, Kyoto-shi (JP); Jumpei Shirono, Yokohama-shi (JP)

(73) Assignee: CANON KABUSHIKI KAISHA, Tokyo (JP)

(21) Appl. No.: 14/218,106

(22) Filed: Mar. 18, 2014

(30) Foreign Application Priority Data
Mar. 26, 2013 (JP) 2013-064203

(51) Int. Cl. A61B 5/00

(52) U.S. Cl. CPC A61B 5/0095
USPC 600/407

(57) ABSTRACT

An object information acquiring apparatus comprises a light source; an acoustic detecting unit configured to detect an acoustic wave generated from an object to which light from the light source is irradiated; and a processing unit configured to acquire characteristic information on the inside of the object based on the acoustic wave and correct the characteristic information using an absorption coefficient of a background region inside the object.

### BACKGROUND OF THE INVENTION

1. Field of the Invention

The present invention relates to an object information acquiring apparatus and an object information acquiring method.

2. Description of the Related Art

Researches on an optical imaging device that allows light emitted to an object from a light source such as a laser to propagate through the inside of the object to obtain internal information on the object are active in medical fields. Japanese Patent Application Publication No. 2010-88627 proposes photoacoustic tomography (PAT) as one of such optical imaging techniques.

PAT is a technique of irradiating an object such as a living body with a pulse beam generated from a light source, detecting acoustic waves generated when light having propagated through and diffused into the living body is absorbed in biological tissues, and analyzing the detected acoustic waves to visualize information on optical characteristics at the inside of the living body. In this way, it is possible to obtain optical characteristic values (in particular, optical energy absorption density) inside the object.

A back-projection method is known as one of reconstruction methods mainly used for calculating initial acoustic pressure. In PAT, initial acoustic pressure P₀ of acoustic waves generated from light absorber in an object can be expressed by Equation (1) below.

P₀=Γ·μₐ·Φ (1)

Here, Γ is a Gruneisen coefficient which is a division of the product of a volume expansion coefficient β and the square of the speed of sound c by the specific heat capacity C_{P} at constant pressure. It is known that δ takes an almost constant value if the object is determined. μₐ is an optical absorption coefficient of the light absorber. Φ is light intensity in a local region (a intensity of light irradiated to the light absorber; also referred to as light fluence).

Japanese Patent Application Publication No. 2010-88627 discloses a technique of measuring the changes over time in acoustic pressure P which is the magnitude of acoustic waves having propagated through the object using an acoustic wave detector and calculating an initial acoustic pressure distribution from the measurement results. According to Japanese Patent Application Publication No. 2010-88627, by dividing the initial acoustic pressure distribution by the Gruneisen coefficient Γ, it is possible to obtain the product of μₐ and Φ (that is, optical energy absorption density).

As expressed in Equation (1), it is necessary to obtain a distribution of light intensity Φ in the object in order to obtain the optical absorption coefficient μₐ from the distribution of the initial acoustic pressure P₀. That is, by dividing the initial acoustic pressure by the light intensity, it is possible to obtain the optical absorption coefficient.

### SUMMARY OF THE INVENTION

However, the initial acoustic pressure obtained by reconstructing measured signals is affected from the frequency-range characteristics of a probe. Thus, it is not possible to obtain an accurate initial acoustic pressure that is based on the product of the absorption coefficient inside the object and the light intensity, as expressed in Equation (1).

As a result, there is a problem that it is difficult to calculate accurate characteristic information such as an absorption coefficient, an oxygen saturation, or a component concentration.

In view of the above problems, it is an object of the present invention to provide a technique of suppressing the influence of frequency-range characteristics of a probe and calculating characteristic information of an object more accurately.

The present invention in its one aspect provides an object information acquiring apparatus comprises a light source; an acoustic detecting unit configured to detect an acoustic wave generated from an object to which light from the light source is irradiated; and a processing unit configured to acquire characteristic information on the inside of the object based on the acoustic wave and correct the characteristic information using an absorption coefficient of a background region inside the object.

The present invention in its another aspect provides a processing method comprises the steps of: acquiring characteristic information on the inside of an object based on an acoustic wave generated from the object to which light is irradiated; and correcting the characteristic information using an absorption coefficient of a background region inside the object.

According to the present invention, it is possible to provide a technique of suppressing the influence of frequency-range characteristics of a probe and calculating characteristic information of an object more accurately.

Further features of the present invention will become apparent from the following description of exemplary embodiments with reference to the attached drawings.

### BRIEF DESCRIPTION OF THE DRAWINGS

FIG. 1 is a diagram illustrating the configuration of an apparatus according to the present invention;

FIG. 2 is a diagram for describing a correction method based on frequency-range sensitivity characteristics of an acoustic wave detector;

FIG. 3 is a flowchart for describing the operation of the present invention;

FIG. 4 is a diagram illustrating the configuration of an apparatus according to a first embodiment;

FIG. 5 is a flowchart for describing the operation of the first embodiment;

FIG. 6 is a diagram illustrating the configuration of an apparatus according to a second embodiment; and

FIG. 7 is a flowchart for describing the operation of the second embodiment.

### DESCRIPTION OF THE EMBODIMENTS

Hereinafter, preferred embodiments of the present invention will be described with reference to the drawings. Dimensions, materials, shapes, relative arrangements, and the like of constituent components described below are to be appropriately changed according to the configuration and various conditions of an apparatus to which the present invention is applied, and the scope of the present invention is not limited to those described below.

The object information acquiring apparatus of the present invention is an apparatus that uses the photoacoustic effect to irradiate an object with light (electromagnetic waves) to transduce an acoustic wave generated in and propagated through the object to thereby acquire object information as image data. For example, the acquired object information may be a generation source distribution of the acoustic wave generated by light irradiation, an initial acoustic pressure distribution inside the object, an optical energy absorption density distribution and an absorption coefficient distribution derived from the initial acoustic pressure distribution, or a concentration distribution of a substance that constitutes a tissue. For example, the substance that constitutes the tissue may be blood components such as an oxygen saturation distribution or an oxygenated or reduced hemoglobin concentration distribution, fats, collagen, or water.

The acoustic wave referred in the present invention is typically an ultrasound wave and includes an elastic wave called a sound wave and an acoustic wave. The acoustic wave generated by the photoacoustic effect is referred to as a photoacoustic wave or a light-induced ultrasound wave. The apparatus according to the present invention transduces an acoustic wave that has been generated in or reflected from the object by an acoustic wave detector such as a probe and has propagated through the object.

(Overview of Apparatus Configuration)

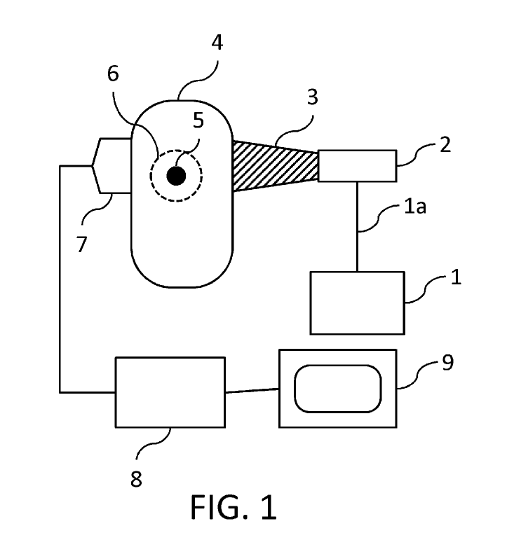

FIG. 1 schematically illustrates an object information acquiring apparatus. A light source 1 is a unit that emits a pulse beam 1a. The pulse beam 1a is irradiated to an object 4 as an irradiation beam 3 by an irradiation optical system 2. When an irradiation beam having propagated through and diffused into the object is absorbed by a light absorber 5, a photoacoustic wave 6 is generated. The photoacoustic wave 6 is received by an acoustic wave detector 7 and is converted into an electrical signal. A processor 8 is a unit that performs information processing such as reconstruction (for example, a process of generating characteristic information of an interest region inside the object from the electrical signal). Image data based on the generated characteristic information is displayed on a monitor 9. The acoustic wave detector corresponds to an acoustic detecting unit of the present invention. The processor corresponds to a processing unit of the present invention.

The processor 8 is a unit that calculates first object information which is internal characteristic information of the object 4 from the electrical signal obtained by the acoustic wave detector 7. The present invention is characterized in that the processor 8 corrects the first object information to calculate second object information.

The first object information is a spatial distribution of characteristic information associated with generation of the photoacoustic wave such as an initial acoustic pressure distribution of the photoacoustic wave generated from the inside of the object 4 or an absorption coefficient distribution calculated based on the initial acoustic pressure distribution. The initial acoustic pressure distribution is generated by the processor 8 applying a back-projection method to the electrical signal output from the acoustic wave detector 7. In this case, it is preferable that the signal output from the acoustic wave detector 7 is converted into a time-series digital signal by an AD converter and is processed by the processor 8. An information processing apparatus such as a PC can be ideally used as the processor 8. The absorption coefficient distribution is calculated using Equation (1) from the initial acoustic pressure distribution calculated in the above-described manner, a light intensity distribution, and a Gruneisen coefficient distribution of an object. Here, the Gruneisen coefficient is a predetermined value corresponding to an object. Moreover, calculated values, estimated values, measured values, and the like can be used for obtaining the light intensity distribution in the object as will be described later.

(Overview of Process Flow)

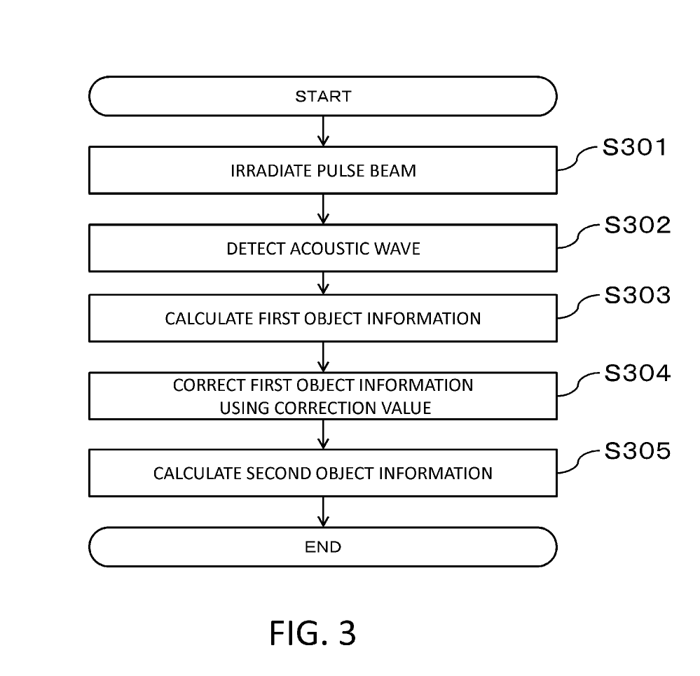

FIG. 3 is a flowchart illustrating an overview of the operation of the present invention.

In step S301, a pulse beam is irradiated to the object 4 from the light source 1. In step S302, the acoustic wave detector 7 provided on the surface of the object receives an acoustic wave generated from the light absorber 5 inside the object to convert the acoustic wave into an electrical signal. In step S303, the processor 8 calculates the first object information on the inside of the object using the electrical signal. The first object information is expressed as characteristic information or a distribution thereof. In step S304, the processor 8 corrects the first object information using a correction value. In step S305, the second object information is calculated based on the corrected first object information.

(Frequency-Range Sensitivity Characteristics)

In the back-projection method, a spatial distribution of the initial acoustic pressure is calculated (reconstructed) from the time-series signals output by the acoustic wave detector 7. Thus, the frequency characteristics of the time-series signals affect the spatial frequency of the calculated spatial distribution. That is, the photoacoustic wave generated from a very small region in a living body is transduced in the acoustic wave detector 7 as a high-frequency signal component and is reconstructed as an initial acoustic pressure distribution (in the very small region) having a high spatial frequency. Moreover, the photoacoustic wave generated from a large region in the living body is transduced in the acoustic wave detector 7 as a low-frequency signal component and is reconstructed as an initial acoustic pressure distribution (in the large region) having a low spatial frequency. When the frequency-range sensitivity characteristics of the acoustic wave detector 7 are different (the acoustic wave reception sensitivity is different from one frequency range to another), this difference affects the reconstructed spatial distribution of the initial acoustic pressure. Practically, an acoustic wave detector often has certain frequency-range sensitivity characteristics.

For example, when the sensitivity of the acoustic wave detector 7 decreases in a high frequency region, a change in the initial acoustic pressure in a small region of the reconstructed initial acoustic pressure distribution is not reproduced (due to a limited resolution). Conversely, when the sensitivity of the acoustic wave detector 7 decreases in a low frequency region, a moderate change in the initial acoustic pressure distribution at the inside of the object is not reproduced. That is, if the sensitivity in the low frequency region is low, the distribution of the initial acoustic pressure which is uniformly distributed inside the object cannot be reproduced according to the back-projection method.

Since the absorption coefficient distribution is calculated based on the initial acoustic pressure distribution, the absorption coefficient distribution is affected by the frequency-range sensitivity characteristics of the acoustic wave detector 7 similarly to the above. Moreover, another reconstruction method which uses the propagation behavior of acoustic waves without limiting to the reconstruction based on the back-projection method is also affected by the frequency-range sensitivity characteristics.

The above-described problem can be rephrased as below. That is, an acoustic wave detector has a low reception sensitivity for acoustic waves of ranges other than its strong range or cannot receive the acoustic waves. If a certain acoustic wave detector has sensitivity characteristics in a high frequency range, although the acoustic wave detector can detect a high-frequency photoacoustic wave generated from a very small light absorber, the acoustic wave detector may hardly (or cannot) detect a low-frequency photoacoustic wave generated from a uniform region (background region) having high homogeneity. That is, it is not possible to detect a low-frequency photoacoustic wave generated from regions (the background region having a background absorption coefficient) other than a light absorber. As a result, even when image reconstruction is performed using the detected signals, the initial acoustic pressure in the background region other than the light absorber cannot be reproduced. Moreover, even at the position of the light absorber, only the difference between the initial acoustic pressure generated from the light absorber and the initial acoustic pressure based on the background absorption coefficient around the light absorber is reproduced.

(Correction Process Based on Correction Value)

In a correction process that characterizes the present invention, correction is performed using a correction value that is based on the frequency-range sensitivity of an acoustic wave detector. This correction value is introduced to solve a problem that a background distribution that changes uniformly or moderately over the entire region of the object cannot be reconstructed due to the frequency-range sensitivity characteristics.

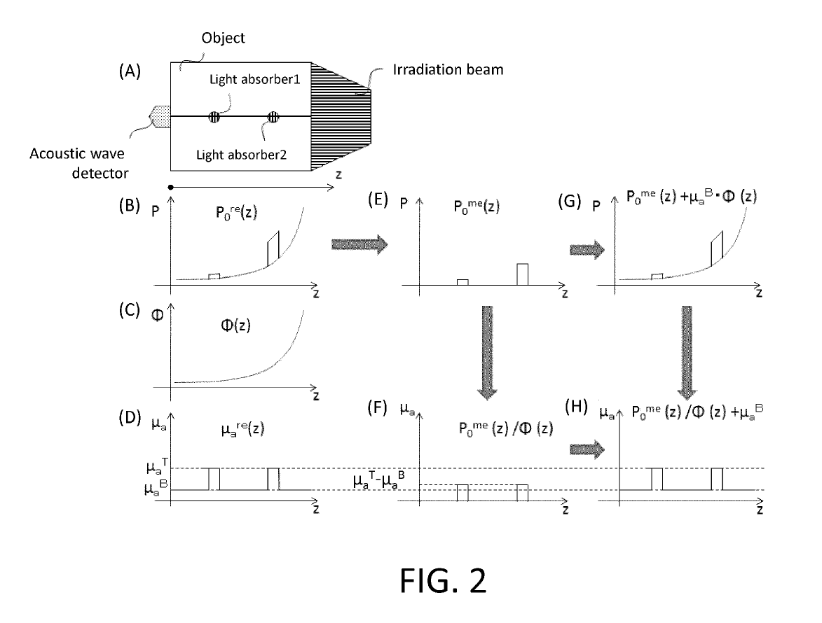

A correction method when the initial acoustic pressure in the background region cannot be observed due to the frequency-range sensitivity characteristics of an acoustic wave detector will be described with reference to FIG. 2.

(A) of FIG. 2 illustrates the configuration for helping description. A sufficiently large area of the object 4 is irradiated with an irradiation beam 3 from the right side. The acoustic wave detector 7 is disposed on an opposite side with a light irradiation surface and the object interposed and is in contact with the object. Light absorbers 5a and 5b are at different depth inside the object. In this example, the light absorbers 5a and 5b are interest regions of the present invention. A background region can be distinguished from the interest regions because the background region has relatively high homogeneity.

(B) of FIG. 2 is an actual initial acoustic pressure distribution P₀^{re}(z) on a line inside the object in (A). The horizontal axis is a depth z from the object surface on which the acoustic wave detector is provided, and the vertical axis is acoustic pressure P. (C) of FIG. 2 is a light intensity distribution Φ(z) on the line inside the object in (A). The horizontal axis is the depth z similarly to the above and the vertical axis is light intensity Φ. (D) of FIG. 2 is an absorption coefficient distribution μₐ^{re}(z) on the line inside the object in (A). The horizontal axis is the depth z and the vertical axis is an absorption coefficient.

As illustrated in (B), the actual initial acoustic pressure is P₀^{re}(z). However, when the acoustic wave detector 7 being used has a central sensitivity range at the frequency range of photoacoustic waves generated from segments having the sizes of the light absorbers 5a and 5b, it is not possible to detect the initial acoustic pressure that depends on the background absorption coefficient. As a result, when the measured initial acoustic pressure is reconstructed, one as illustrated in (E) is obtained. (E) of FIG. 2 is an initial acoustic pressure distribution P₀^{me}(z) obtained by reconstructing signals detected by the acoustic wave detector. The horizontal axis is the depth z and the vertical axis is acoustic pressure P. When (E) and (B) are compared, the background absorption coefficient component is deficient in (E).

That is, if the background absorption coefficient of the object 4 is μₐ^{B}, the absorption coefficient of a light absorber is μₐ^{T}, the position of the light absorber 5a is z^{T1}, the position of the light absorber 5b is z^{T2}, and the position of the background is z^{B}, the initial acoustic pressures at the respective positions are expressed by Equations (2a) to (2c) and Equations (3a) to (3c).

P₀^{re}(z^{T1})=μₐ^{T}·Φ(z^{T1}) (2a)

P₀^{re}(z^{T2})=μₐ^{T}·Φ(z^{T2}) (2b)

P₀^{re}(z^{B})=μₐ^{B}·Φ(z^{B}) (2c)

P₀^{me}(z^{T2})=(μₐ^{T}−μₐ^{B})·Φ(z^{T1}) (3a)

P₀^{me}(z^{T1})=(μₐ^{T}−μₐ^{B})·Φ(z^{T2}) (3b)

P₀^{me}(z^{B})=(μₐ^{B}−μₐ^{B})·Φ(z^{B}) (3c)

Thus, even when the initial acoustic pressure P₀^{me}(z) obtained by reconstruction is divided by Φ(z), an accurate absorption coefficient distribution is not obtained but a distribution as illustrated in (F) is obtained. (F) of FIG. 2 illustrates P₀^{me}(z)/Φ(z) obtained by dividing the initial acoustic pressure P₀^{me}(z) of (E) by the light intensity Φ(z). The horizontal axis is the depth z from the object surface on which the acoustic wave detector is provided, and the vertical axis is the absorption coefficient. The absorption coefficient of (F) is lower than the actual absorption coefficient distribution ((D) of FIG. 2) by the background absorption coefficient μ^{B}. As above, if the central sensitivity range of the acoustic wave detector is fitted to the size of a light absorber in the object, since it is not possible to sufficiently receive the acoustic wave from the background region of the object, the reconstructed initial acoustic pressure or the calculated absorption coefficient is inaccurate.

Thus, in the present invention, by adding the background absorption coefficient to the absorption coefficient obtained in (F), it is possible to calculate an accurate absorption coefficient as illustrated in (H) of FIG. 2. In (H), the horizontal axis is the depth z and the vertical axis is an absorption coefficient.

Moreover, as illustrated in (G) of FIG. 2, a method of adding an initial acoustic pressure μₐ^{B}·Φ(z) associated with the background absorption coefficient to the initial acoustic pressure P₀^{me}(z) of (E) may be used. By dividing the addition result by Φ(z), it is possible to calculate an accurate absorption coefficient distribution P₀^{me}(z)/Φ(z)+μₐ^{B}.

For example, the background absorption coefficient can be acquired by time-resolved spectroscopy or frequency-resolved spectroscopy. That is, the processor 8 can acquire a background absorption coefficient from a detection signal of light having propagated through an object. Moreover, the background absorption coefficient can be acquired by being input by an input unit.

For example, it is assumed that the absorption coefficients μₐ^{are}(z^{T1}) and μₐ^{re}(z^{T2}) of the light absorbers 5a and 5b are 0.012/mm and 0.009/mm, respectively, and the background absorption coefficient μₐ^{re} (z^{B}) is 0.005/mm. Moreover, it is assumed that the light quantities Φ(z^{T1}) and Φ(z^{T2}) at the positions of the light absorbers 5a and 5b are 50 mJ/mm² and 900 mJ/mm², respectively. In this case, the actual initial acoustic pressures at the positions of the light absorbers 5a and 5b are 0.6 mJ/mm³ and 8.1 mJ/mm³, respectively.

On the other hand, the initial acoustic pressures (measured values) obtained by reconstructing detection signals detected using a probe of which the central range is fitted to the frequency range corresponding to the sizes of these light absorbers are 0.35 mJ/mm³ and 3.6 mJ/mm³, respectively. The absorption coefficients calculated by dividing the initial acoustic pressures obtained by reconstruction by the light quantities at the respective positions are 0.007/mm and 0.004/mm. Thus, the absorption coefficients are considerably smaller than the actual absorption coefficients, and it is not possible to calculate the accurate absorption coefficients.

Thus, when the background absorption coefficient (0.005/mm) is added to these absorption coefficients (based on the measured value) according to the method of the present invention, the absorption coefficients of the light absorbers 5a and 5b are 0.012/mm and 0.009/mm, respectively, and the accurate values can be calculated. Moreover, when the values (0.25 mJ/mm² and 4.5 mJ/mm², respectively) obtained by multiplying the background absorption coefficient and the light quantities are added to the initial acoustic pressures 0.35 mJ/mm³ and 3.6 mJ/mm³ of the light absorbers 5a and 5b obtained by the reconstruction, values 0.6 mJ/mm² and 8.1 mJ/mm² are obtained. By dividing these values by the light intensity, it is possible to calculate accurate absorption coefficients of 0.012/mm and 0.009/mm.

### First Embodiment

In the present embodiment, an object information acquiring apparatus that images an oxygen saturation distribution in the breast as an object will be described. In the present embodiment, the object is held by being interposed between parallel flat holding plates. Moreover, the object information acquiring apparatus of the present embodiment includes a frequency-resolved spectroscopy measurement mechanism and calculates a background absorption coefficient of the object by frequency-resolved spectroscopy to use the background absorption coefficient as a correction value.

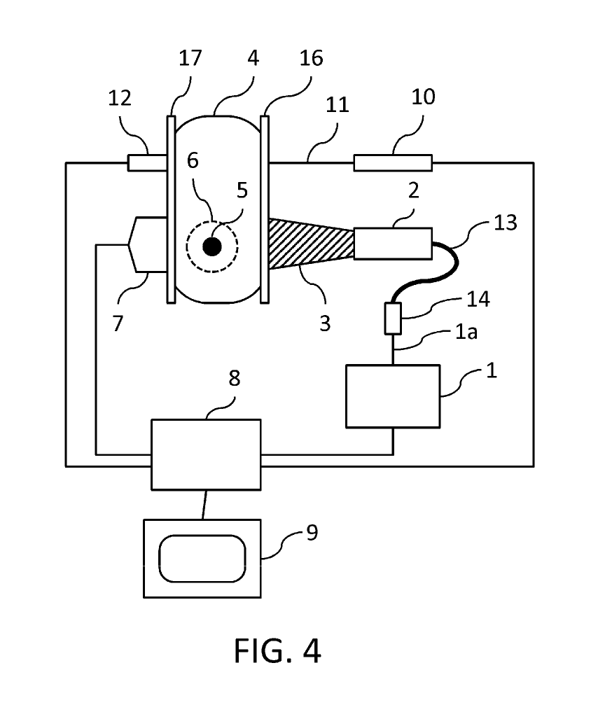

The configuration of the apparatus of the present embodiment will be described with reference to FIG. 4. Only those components different from those of FIG. 1 will be described. Holding plates 16 and 17 are formed of polymethylpentene that is transmissive to both light and acoustic waves. The holding plates 16 and 17 are parallel flat plates and at least one is movable. The object 4 can be interposed and fixed between the holding plates 16 and 17 when the gap therebetween changes.

First, in the object information acquiring apparatus of the present embodiment, a mechanism for calculating the background absorption coefficient of the object 4 will be described. A second light source 10 and a photodetector 12 are disposed over the holding plates with the object interposed so as to face each other. The second light source 10 can emit light of a plurality of wavelengths. That is, the second light source 10 can irradiate the object by switching laser diodes having wavelengths of 637 nm, 686 nm, 756 nm, 797 nm, 808 nm, 852 nm, 912 nm, and 975 nm. The photodetector 12 is an avalanche photodiode and detects a component of an irradiation beam 11 which has been emitted from the second light source 10, diffused into the object 4, and reached the position of the photodetector 12. The processor 8 inputs a time waveform of an application voltage for outputting light from a laser diode as a reference waveform and an intensity waveform of light detected by an avalanche photodiode as a measurement waveform to a lock-in amplifier and calculates a phase change and light intensity attenuation. The processor 8 calculates a background absorption coefficient and a background scattering coefficient using the calculated phase change and light intensity attenuation. The background absorption coefficient and the background scattering coefficient are calculated by solving an inverse problem of light propagation assuming that the absorption coefficient and the scattering coefficient of the object 4 are constant. Further, the processor 8 performs fitting with respect to the background absorption coefficients and the background scattering coefficients at the respective wavelengths of the object 4 using the absorption spectrums of fats, water, oxygenated hemoglobin, and reduced hemoglobin to calculate respective component amounts. In FIG. 4, the second light source 10 is provided separately from the photoacustic measurement light source 1. However, the photodetector 12 may detect light that has been emitted from the light source 1 and passed through the irradiation optical system 2. Moreover, the photodetector 12 may detect light that is branched from the light source 1 by an optical fiber or the like. The photodetector corresponds to alight detecting unit of the present invention.

Next, in the object information acquiring apparatus of the present embodiment, a mechanism for performing photoacoustic measurement using light having wavelengths of 756 nm and 797 nm to calculate the absorption coefficient distributions of the respective wavelengths, and correcting the absorption coefficient distributions using a background absorption coefficient distribution to calculate an oxygen saturation will be described. By obtaining the absorption coefficients at a plurality of wavelengths, it is possible to calculate a concentration of a substance.

The irradiation optical system 2 and the acoustic wave detector 7 are disposed with the object interposed so as to face each other to perform transmissive photoacoustic measurement.

The light source 1 is a titanium-sapphire laser capable of emitting light of 756 nm and 797 nm. A pulse beam 1a of the wavelength 756 nm emitted from the light source 1 enters a bundle fiber 13 through a bundle fiber-incident optical system 14. An output end of the bundle fiber 13 is connected to the irradiation optical system 2, and a pulse beam emitted from the fiber is irradiated to the object 4 as an irradiation beam 3 through a lens, a diffuser, and the like so that irradiation wavelength dependence decreases.

The irradiation beam 3 is diffused into the object 4 and absorbed by the light absorber 5, and the photoacoustic wave 6 is generated.

The photoacoustic wave 6 is converted into electrical signals by the acoustic wave detector 7. The acoustic wave detector 7 is a 2-dimensional array transducer and is formed of a piezoelectric element having a central frequency of 2 MHz. The processor 8 calculates an initial acoustic pressure distribution P₀ (756 nm, r) at the wavelength 756 nm using the electrical signals according to the back-projection method. The processor 8 solves a light diffusion equation by the finite element method using the object shape measured in advance, the distribution of the irradiation beam 3, the background absorption coefficient, and the background scattering coefficient to calculate the light intensity distribution Φ (756 nm, r) at the wavelength 756 nm. The processor 8 calculates the absorption coefficient μₐ (756 nm, r) using Equation (1) based on the initial acoustic pressure P₀ (756 nm, r) and the light intensity Φ (756 nm, r) calculated in this manner. Further, the processor 8 calculates a correction target absorption coefficient distribution μₐ^{C} (756 nm, r) by adding the background absorption coefficient μₐ^{B} (756 nm) obtained by frequency-resolved spectroscopy to the absorption coefficient distribution μₐ (756 nm, r).

Similarly, the pulse beam 1a having the wavelength 797 nm emitted from the light source 1 is irradiated to the object 4 as the irradiation beam 3 through the bundle fiber-incident optical system 14, the irradiation optical system 2 connected to the output end of the bundle fiber 13, the lens, the diffuser, and the like. The irradiation beam 3 is diffused into the object 4 and absorbed by the light absorber 5, and the photoacoustic wave 6 is generated. The photoacoustic wave 6 is converted into electrical signals by the acoustic wave detector 7. The acoustic wave detector 7 is a 2-dimensional array transducer and is formed of a piezoelectric element having a central frequency of 2 MHz. The processor 8 calculates an initial acoustic pressure distribution P₀ (797 nm, r) at the wavelength 797 nm using the electrical signals according to the back-projection method. The processor 8 solves a light diffusion equation by the finite element method using the object shape measured in advance, the distribution of the irradiation beam 3, the background absorption coefficient, and the background scattering coefficient to calculate the light intensity distribution Φ (797 nm, r) at the wavelength 797 nm. The processor 8 calculates the absorption coefficient μₐ (797 nm, r) using Equation (1) based on the initial acoustic pressure P₀ (797 nm, r) and the light intensity Φ (797 nm, r) calculated in this manner. Further, the processor 8 calculates a correction target absorption coefficient distribution μₐ^{C} (797 nm, r) by adding the background absorption coefficient μₐ^{B} (797 nm) obtained by frequency-resolved spectroscopy to the absorption coefficient distribution μ^{a} (797 nm, r).

The processor 8 calculates the oxygen saturation using Equation (4) based on the correction target absorption coefficient distributions μₐ^{C} (756 nm, r) and μₐ^{C} (797 nm, r) at the respective wavelengths.

$$\begin{matrix} [ Math . 1 ] & \\ {StO}_{2} ( r ) = \frac{- {μ}_{a}^{C} ( 756 nm , r ) {ɛ}_{Hb} ( 797 nm ) + {μ}_{a}^{C} ( 756 nm , r ) {ɛ}_{Hb} ( 756 nm )}{\begin{matrix}- {μ}_{a}^{C} ( 756 nm , r ) \{ {ɛ}_{Hb} ( 797 nm ) - {ɛ}_{HbO} ( 797 nm ) \} + \\ {μ}_{a}^{C} ( 797 nm , r ) \{ {ɛ}_{Hb} ( 756 nm ) - {ɛ}_{HbO} ( 756 nm ) \}\end{matrix}} & ( 4 )\end{matrix}$$

Here, ε_{Hb}(λ) and ε_{Hb0}(λ) are the absorption coefficients of reduced and oxygenated hemoglobin at the respective wavelengths λ (nm).

The calculated oxygen saturation distribution is displayed on the monitor 9 in a form of a 3-dimensional image, slice images, or the like.

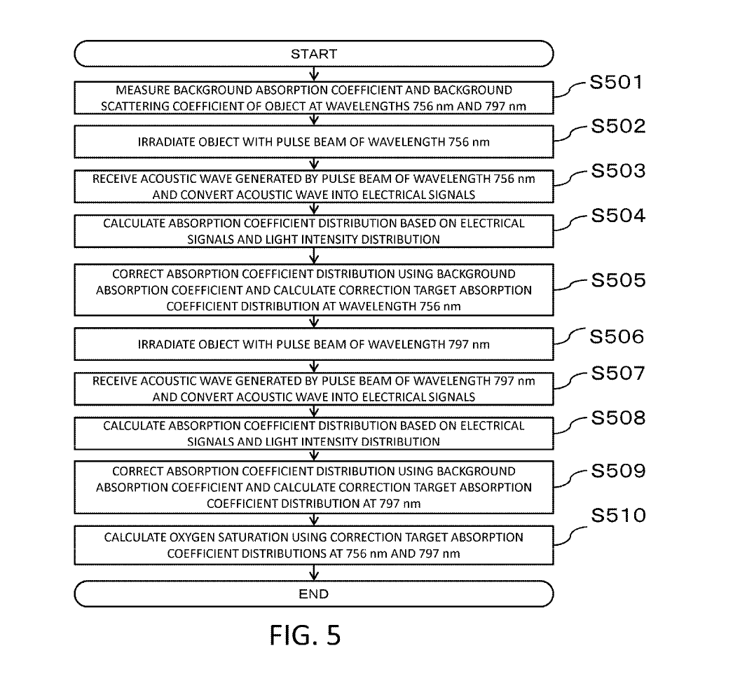

FIG. 5 illustrates a flowchart of the present embodiment. The method of the present embodiment will be described with reference to FIG. 5.

First, in step S501, frequency-resolved spectroscopy is performed to calculate the background absorption coefficients and background scattering coefficients at the wavelengths 756 nm and 797 nm.

Subsequently, the flow proceeds to measurement at the wavelength 756 nm. In step S502, a pulse beam of the wavelength 756 nm is irradiated to an object. In step S503, the acoustic wave detector detects an acoustic wave generated from a light absorber when the pulse beam of the wavelength 756 nm is irradiated and converts the acoustic wave into electrical signals. In step S504, an initial acoustic pressure distribution at the wavelength 756 nm is calculated based on the electrical signals, and the light intensity distribution is calculated based on the object shape measured in advance, the irradiation beam distribution, and the background absorption coefficient and the background scattering coefficient of the object at the wavelength 756 nm to calculate the absorption coefficient distribution. In step S505, a correction target absorption coefficient distribution at the wavelength 756 nm is calculated by adding the background absorption coefficient to the absorption coefficient distribution. This step is a step of “correcting characteristic information” that characterizes the present invention.

Subsequently, the flow proceeds to measurement at the wavelength 797 nm. In step S506, a pulse beam of the wavelength 797 nm is irradiated to an object. In step S507, the acoustic wave detector detects an acoustic wave generated from a light absorber when the pulse beam of the wavelength 797 nm is irradiated and converts the acoustic wave into electrical signals. In step S508, an initial acoustic pressure distribution at the wavelength 797 nm is calculated based on the electrical signals, and the light intensity distribution is calculated based on the object shape measured in advance, the irradiation beam distribution, and the background absorption coefficient and the background scattering coefficient of the object at the wavelength 797 nm to calculate the absorption coefficient distribution. In step S509, a correction target absorption coefficient distribution at the wavelength 797 nm is calculated by adding the background absorption coefficient to the absorption coefficient distribution. This step is a correction step based on a correction value.

Finally, in step S510, the oxygen saturation distribution is calculated according to Equation (2) based on the correction target absorption coefficient distributions at the wavelengths 756 nm and 797 nm.

As described above, due to the frequency-range sensitivity characteristics of the acoustic wave detector, it may be not possible to sufficiently receive acoustic waves from the background region and to obtain an accurate initial acoustic pressure (or the absorption coefficient, the oxygen saturation, or the like obtained therefrom). However, according to the present invention, even in such cases, it is possible to correct the measured initial acoustic pressure or the calculated absorption coefficient using a correction value based on the background absorption coefficient and the background scattering coefficient to obtain more accurate values and use the values in generating image data and diagnosis.

The present invention can be understood as an object information acquiring apparatus as described above. Moreover, the present invention can be understood as a control method of the apparatus. Further, the present invention can be realized as a program for causing a processor or the like of the apparatus to execute the respective steps of the control method.

### Second Embodiment

In the present embodiment, an object information acquiring apparatus that images an oxygen saturation distribution in the breast as an object will be described. In the present embodiment, the object is inserted in a water tank and is measured. Moreover, the object information acquiring apparatus of the present embodiment includes an input unit, and a background absorption coefficient measured or estimated in advance is input and used as a correction value.

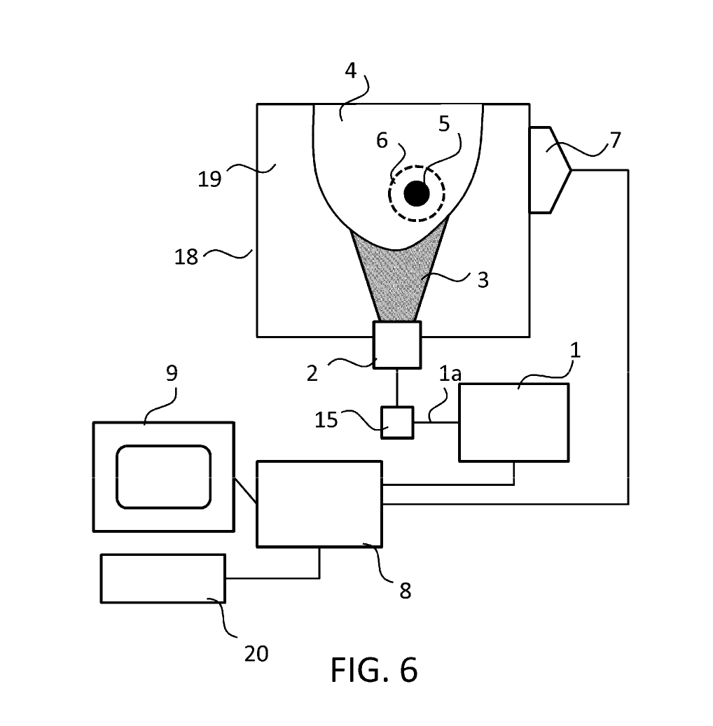

The configuration of the apparatus according to the present embodiment will be described with reference to FIG. 6. Only those components different from those of FIG. 1 will be described. A container 18 formed of polymethylpentene that is transmissive to both light and acoustic waves. The container 18 has an open upper surface (the upper side of the drawing sheet) and is filled with water 19. The object 4 is inserted in the container 18 from the upper surface and is sunk into the water 19.

A frequency spectroscopy measurement mechanism is incorporated into the apparatus of the first embodiment, and the background absorption coefficient and the background scattering coefficient of the object 4 are calculated using the frequency spectroscopy measurement mechanism. In contrast, the apparatus of the present embodiment includes an input unit 20, and the background absorption coefficient and the background scattering coefficient of the object 4 are acquired when an operator of the present apparatus operates the input unit 20 to input the background absorption coefficient and the background scattering coefficient of the object 4. The input background absorption coefficient and background scattering coefficient may be input as values that are measured by another apparatus and values that are estimated using an age, a BMI value, and the like. If the background absorption coefficient and the background scattering coefficient of the object 4 are known, the known values may be input. Moreover, the age, the BMI value, and the like that enable the background absorption coefficient and the background scattering coefficient to be estimated may be input. In this case, the processor 8 can calculate the background absorption coefficient and the background scattering coefficient based on the input age and BMI value, and the like. Moreover, the processor 8 may read and acquire the background absorption coefficient and the background scattering coefficient corresponding to the input age and BMI value, and the like from a data table.

Next, in the object information acquiring apparatus of the present embodiment, a mechanism for performing photoacoustic measurement using light having a wavelength of 1064 nm to calculate the initial acoustic pressure distribution, and correcting the initial acoustic pressure distribution using the background absorption coefficient and the light intensity distribution to calculate a correction target initial acoustic pressure distribution will be described. The irradiation optical system 2 is disposed to irradiate the object 4 from the below. The acoustic wave detector 7 is disposed on a side surface of the water tank 18 so that the object 4 can be measured from all directions(360°) and that the photoacoustic wave 6 generated from the light absorber 5 of the object 4 can scan the wall surfaces of the water tank 18.

The light source 1 is a YAG laser that can emit light of 1064 nm. The pulse beam 1a of the wavelength 1064 nm emitted from the light source 1 enters an air-propagation optical system 15. Since the output end of the air-propagation optical system 15 is connected to the irradiation optical system 2 and the irradiation beam 3 is uniformly irradiated to a certain range of the object 4, the pulse beam 1a is irradiated to the object 4 as the irradiation beam 3 through a lens and a diffuser.

The irradiation beam 3 is diffused into the object 4 and absorbed by the light absorber 5, and the photoacoustic wave 6 is generated.

The photoacoustic wave 6 is converted into electrical signals by the acoustic wave detector 7. The acoustic wave detector 7 is a 1-dimensional array transducer and is formed of a cMUT element having a central frequency of 3 MHz. The processor 8 calculates an initial acoustic pressure distribution P₀ (1064 nm, r) of the light having the wavelength 1064 nm using the electrical signals according to a sequential reconstruction method. The processor 8 solves a phototransport equation by the finite element method using the object shape measured in advance, the distribution of the irradiation beam 3, the background absorption coefficient, and the background scattering coefficient to calculate the light intensity distribution Φ (1064 nm, r) of the light having the wavelength 1064 nm. The processor 8 calculates a correction target initial acoustic pressure distribution P₀^{c} (1064 nm, r) by adding the product P₀^{B} (1064 nm, r) of the light intensity Φ (1064 nm, r) and the background absorption coefficient μLₐ^{B} (1064 nm) to the initial acoustic pressure P₀ (1064 nm, r) calculated in this way.

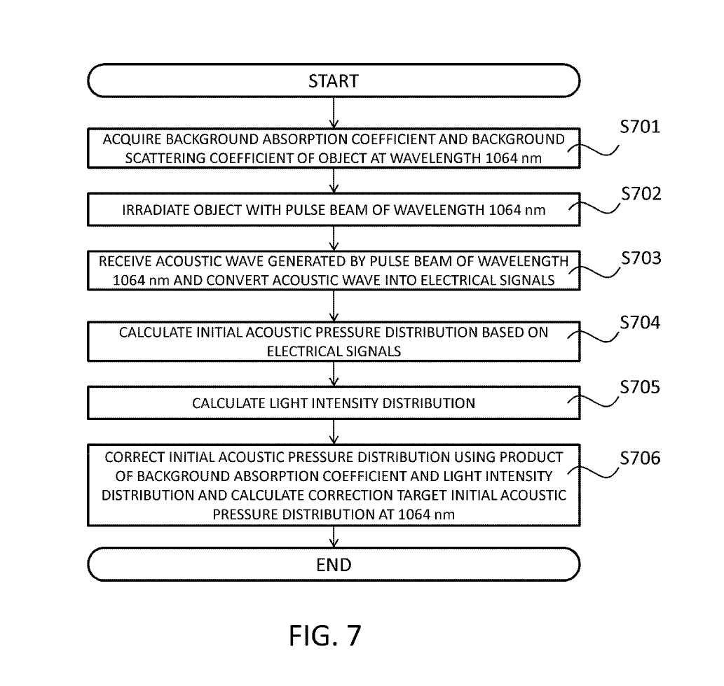

FIG. 7 illustrates a flowchart of the present embodiment. The method of the present embodiment will be described with reference to FIG. 7.

First, in step S701, the background absorption coefficient and the background scattering coefficient at the wavelength 1064 nm input by the input unit are acquired.

Subsequently, the flow proceeds to measurement at the wavelength 1064 nm. In step S702, a pulse beam of the wavelength 1064 nm is irradiated to an object. In step S703, the acoustic wave detector detects an acoustic wave generated from a light absorber when the pulse beam of the wavelength 1064 nm is irradiated and converts the acoustic wave into electrical signals. In step S704, an initial acoustic pressure distribution at the wavelength 1064 nm is calculated based on the electrical signals. In step S705, the light intensity distribution is calculated based on the object shape measured in advance, the irradiation beam distribution, and the background absorption coefficient and the background scattering coefficient of the object at the wavelength 1064 nm. In step S706, a correction target initial acoustic pressure distribution at the wavelength 1064 nm is calculated by adding the product of the background absorption coefficient and the light intensity distribution to the initial acoustic pressure distribution. This step is a step of “correcting characteristic information” that characterizes the present invention.

As described above, due to the frequency-range sensitivity characteristics of the acoustic wave detector, it may be not possible to sufficiently receive acoustic waves from the background region and to obtain an accurate initial acoustic pressure (or the absorption coefficient, the oxygen saturation, or the like obtained therefrom). However, according to the present invention, even in such cases, it is possible to correct the measured initial acoustic pressure or the calculated absorption coefficient using a correction value based on the background absorption coefficient and the background scattering coefficient to obtain more accurate values and use the values in generating image data and diagnosis.

The present invention can be understood as an object information acquiring apparatus as described above. Moreover, the present invention can be understood as a control method of the apparatus. Further, the present invention can be realized as a program for causing a processor or the like of the apparatus to execute the respective steps of the control method.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-064203, filed on Mar. 26, 2013, which is hereby incorporated by reference herein in its entirety.

What is claimed is:

1. An object information acquiring apparatus comprising:
a light source;
an acoustic detecting unit configured to detect an acoustic wave generated from an object to which light from the light source is irradiated; and
a processing unit configured to acquire characteristic information on the inside of the object based on the acoustic wave and correct the characteristic information using an absorption coefficient of a background region inside the object.

2. The object information acquiring apparatus according to claim 1, further comprising:
a second light source;
a light detecting unit configured to detect light that has been emitted from the second light source and has propagated through the object, wherein
the processing unit acquires the absorption coefficient of the background region based on an intensity of the light detected by the light detecting unit.

3. The object information acquiring apparatus according to claim 2, wherein
the processing unit acquires the absorption coefficient of the background region by time-resolved spectroscopy or frequency-resolved spectroscopy based on the intensity of the light detected by the light detecting unit.

4. The object information acquiring apparatus according to claim 1, further comprising:
an input unit configured to input the absorption coefficient of the background region.

5. The object information acquiring apparatus according to claim 1, further comprising:
an input unit configured to input information that enables the absorption coefficient of the background region to be estimated, wherein
the processing unit acquires the absorption coefficient of the background region based on the information.

6. The object information acquiring apparatus according to claim 5, wherein
the input unit is configured to input an age or a BMI as the information.

7. The object information acquiring apparatus according to claim 1, wherein
the processing unit is configured to:
acquire an initial acoustic pressure distribution at the inside of the object as the characteristic information based on the acoustic wave;
acquire a light intensity distribution at the inside of the object, of the light from the light source;
acquire an initial acoustic pressure of the background region using the absorption coefficient of the background region and the light intensity distribution; and
correct the initial acoustic pressure distribution using the initial acoustic pressure of the background region.

8. The object information acquiring apparatus according to claim 7, wherein
the processing unit is configured to:
correct the initial acoustic pressure distribution by acquiring sum of the initial acoustic pressure distribution and the initial acoustic pressure of the background region.

9. The object information acquiring apparatus according to claim 1, wherein
the processing unit is configured to:
acquire an initial acoustic pressure distribution at the inside of the object based on the acoustic wave;
acquire a light intensity distribution at the inside of the object, of the light from the light source;
acquire an absorption coefficient distribution at the inside of the object as the characteristic information based on the initial acoustic pressure distribution and the light intensity distribution; and
correct the absorption coefficient distribution using the absorption coefficient of the background region.

10. The object information acquiring apparatus according to claim 9, wherein
the processing unit is configured to:
correct the absorption coefficient distribution by acquiring sum of the absorption coefficient distribution and the absorption coefficient of the background region. n.

11. The object information acquiring apparatus according to claim 1, wherein
the background region is a region in which a spatial frequency of the generated acoustic wave is lower than that of a light absorber which is an interest region, at the inside of the object, in which an absorption coefficient is high.

12. The object information acquiring apparatus according to claim 1, wherein
the background region is a region in which homogeneity is higher than that of a light absorber inside the object.

13. The object information acquiring apparatus according to claim 2, wherein
the light source also serves as the second light source.

14. The object information acquiring apparatus according to claim 1, further comprising:
a monitor configured to display the characteristic information.

15. A processing method comprising the steps of:
acquiring characteristic information on the inside of an object based on an acoustic wave generated from the object to which light is irradiated; and
correcting the characteristic information using an absorption coefficient of a background region inside the object.

16. The processing method according to claim 15, wherein
the step of acquiring the characteristic information includes acquiring an initial acoustic pressure distribution at the inside of the object as the characteristic information based on the acoustic wave, and
the step of correcting the characteristic information includes acquiring a light intensity distribution at the inside of the object, of the light from the light source, acquiring an initial acoustic pressure of the background region using the absorption coefficient of the background region and the light intensity distribution, and correcting the initial acoustic pressure distribution using the initial acoustic pressure of the background region.

17. The processing method according to claim 16, wherein
the step of correcting the characteristic information includes summing the initial acoustic pressure distribution and the initial acoustic pressure of the background region.

18. The processing method according to claim 15, wherein
the step of acquiring the characteristic information includes acquiring an initial acoustic pressure distribution at the inside of the object, acquiring a light intensity distribution at the inside of the object, of the light from the light source, and acquiring an absorption coefficient distribution at the inside of the object as the characteristic information based on the initial acoustic pressure distribution and the light intensity distribution, and
the step of correcting the characteristic information includes correcting the absorption coefficient distribution using the absorption coefficient of the background region.

19. The processing method according to claim 18, wherein
the step of acquiring the characteristic information includes summing the absorption coefficient distribution and the absorption coefficient of the background region.
